(19) United States
(12) Patent Application Publication
(10) Pub. No.: US 2009/0306422 A1
(43) Pub. Date: Dec. 10, 2009

(54) OLIGOMERIC URETHANE ACRYLATES, THEIR PREPARATION AND USE

(75) Inventors: Maxime Allard, Dachstein (FR); Heinz-Peter Rink, Munster (DE); Werner-Alfons Jung, Ascheberg (DE); Jennifer Moebius, Enschede (NL)

Correspondence Address: Mary E. Golota;Cantor Colburn LLP, 201 W. Big Beaver Road, Suite 1101, Troy, MI, 48084, US

(73) Assignee: BASF Coatings AG, Munster (DE)

(21) Appl. No.: 12/162,513

(22) PCT Filed: Feb. 9, 2007
(86) PCT No.: PCT/EP07/01142

§ 371 Date: Feb. 4, 2009

(30) Foreign Application Priority Data
Feb. 11, 2006 (DE) 10 2006 006 334.1

(51) Int. Cl. C07C 271/06

(52) U.S. Cl. 560/158

(57) ABSTRACT

An oligomeric urethane acrylate composition is disclosed. The composition comprises, on average per molecule, at least two structural units of the general formula I: R{—X—CH₂—CH(—CH₂—O—(O)—CR¹═CH₂)[—O—C(O)—NH—]}ₙ (I), where n is a number from 1 to 6, R is a monovalent to hexavalent organic radical, X is oxygen atom or —C(O)—O— radical linked by the carbon atom to the radical R, and R¹ is hydrogen atom, halogen atom, nitrile group, substituted or unsubstituted alkyl group having 1 to 6 carbon atoms, substituted or unsubstituted cycloalkyl group having 3 to 6 carbon atoms, or substituted or unsubstituted aryl group having 6 to 10 carbon atoms. Also disclosed are processes for preparing and using the compositions.

The present invention relates to new oligomeric urethane acrylates. The present invention also relates to a new process for preparing oligomeric urethane acrylates. The present invention relates not least to the use of the new oligomeric urethane acrylates and of the oligomeric urethane acrylates prepared by the new process as or to prepare materials which are free-radically curable thermally and/or with actinic radiation.

### PRIOR ART

Oligomeric urethane acrylates which can be activated by actinic radiation, processes for preparing them, and their use for producing materials which are free-radically curable thermally and/or with actinic radiation are known from German patent application DE 199 15 070 A1.

Here and below, actinic radiation means electromagnetic radiation such as near infrared (NIR), visible light, UV radiation, X-rays, and gamma radiation, especially UV radiation, and particulate radiation such as electron beams, proton beams, alpha radiation, beta radiation, and neutron beams, especially electron beams.

The known oligomeric urethane acrylates are prepared by preparing in a first stage a carboxyl-containing polyester from (meth)acrylic acid, a polyhydric alcohol, trimethylolpropane for example, and a polycarboxylic acid, adipic acid for example. The resulting carboxyl-containing polyester is reacted with a monoepoxide compound or a polyfunctional epoxide compound, such as bisphenol A diglycidyl ether. The resulting polyesters, which contain secondary hydroxyl groups, are reacted with polyisocyanates to give the known urethane acrylates. These acrylates contain, accordingly, structural units such as, for instance

—C₆H₄—O—CH₂—CH[—O—(O)C-polyester(—O—(O)C—CH═CH₂)ₓ][—O—(O)C—NH-]

with x≧1.

The known oligomeric urethane acrylates have a comparatively high viscosity. Consequently, in order to prepare coating materials which can be applied, it is necessary to admix them with organic solvents and/or reactive diluents which can be activated with actinic radiation.

The addition of reactive diluents also becomes necessary if pigments and/or flatting agents are to be incorporated into the known curable materials in question, particularly with the aim of obtaining coating materials which can be applied by the coil-coating method and are intended to produce pigmented and/or flatted coatings, such as flat or silkily glossy white topcoats, on coils. Additionally, the preparation of clearcoat materials for producing glossily clear primer coatings and topcoats or clearcoats is generally not possible without reactive diluents.

Adding the reactive diluents, however, may have deleterious consequences. In particular the reactive diluents may result in a polymerization-associated contraction in the course of curing, which adversely affects the profile of properties of the resulting coatings. Mechanical properties as well, such as the flexibility so essential for the deformability of coated coils, the chemical resistance, the weathering stability, and the adhesion, particularly on coils, may suffer.

The addition of organic solvents as well is a disadvantage, because during the preparation, application, and curing of the known coating materials these solvents must be evaporated off, worked up, and disposed of, all at some cost and inconvenience.

### PROBLEM ADDRESSED BY THE INVENTION

It is an object of the present invention to provide new oligomeric urethane acrylates which can be activated with actinic radiation and have a low viscosity. Their viscosity in the DIN 6 flow cup at 23° C. ought to be <500 s, preferably <450 s, and in particular <400 s.

The new oligomeric urethane acrylates ought to be preparable easily, economically, and with very good reproducibility from readily available starting products.

The new oligomeric urethane acrylates ought to be outstandingly suitable as or for preparing materials which are free-radically curable thermally and/or with actinic radiation. In this context they ought to be able to be mixed without problems and without great expenditure of energy with customary and known additives, especially pigments and flatting agents.

The new materials free-radically curable thermally and/or with actinic radiation ought to be outstandingly suitable for producing new thermoset materials having a very good profile of properties. In particular they ought to be outstandingly suitable as new coating materials, adhesives, sealants, and precursors for moldings and sheets, all free-radically curable thermally and/or with actinic radiation, for producing new thermoset coatings, adhesive layers, seals, moldings, and sheets.

In particular it is the intention that the new coating materials free-radically curable thermally and/or with actinic radiation should be able to be applied without problems, using rapid methods, to a wide variety of substrates, even without reactive diluent or with only a very small amount of reactive diluents, and also without organic solvent or with only a very small amount of organic solvents. In particular they ought to be able to be applied to coils by the coil-coating method without problems.

The applied new coating materials free-radically curable thermally and/or with actinic radiation ought to be able to be free-radically cured thermally and/or with actinic radiation, rapidly and without polymerization-induced contraction, or with such little polymerization-induced contraction that the desired profile of properties is not affected, or not markedly affected, and ought to give new thermoset coatings, especially glossily clear transparent and flat transparent primer coatings, glossy opaque and flat opaque basecoats, glossily clear transparent and flat transparent topcoats, and glossy opaque and flat opaque topcoats, all having an outstanding profile of properties.

In particular the new thermoset coatings ought to exhibit very good mechanical properties, in particular a high hardness, flexibility, and deformability, strong adhesion to a wide variety of substrates, especially to coils, and also high chemical resistance and weathering stability. The new flat thermoset coatings ought to have an outstanding flatting effect through to a silk gloss.

### SOLUTION PROVIDED BY THE INVENTION

Found accordingly have been the new oligomeric urethane acrylates containing on average per molecule at least two structural units of the general formula I:

R{-X—CH₂—CH(—CH₂—O—(O)—CR¹═CH₂)[—O—C(O)—NH-]}ₙ (I),

in which the index and variables are defined as follows:
n is a number from 1 to 6;
R is monovalent to hexavalent, low molecular mass or oligomeric, organic radical;
X is oxygen atom or —C(O)—O— radical linked by the carbon atom to the radical R; and
R¹ is hydrogen atom, halogen atom, nitrile group, substituted or unsubstituted alkyl group having 1 to 6 carbon atoms, substituted or unsubstituted cycloalkyl group having 3 to 6 carbon atoms, or substituted or unsubstituted aryl group having 6 to 10 carbon atoms.

The new oligomeric urethane acrylates are referred to below as “urethane acrylates of the invention”.

Also found has been the new process for preparing urethane acrylates of the invention, which involves reacting at least one compound of the general formula III:

R[—X—CH₂—CH(—OH)(—CH₂—O—(O)—CR¹═CH₂)]ₙ (III),

in which the index n and the variables R, X, and R¹ are as defined above, with at least one polyisocyanate having at least two isocyanate groups, in a compound III: polyisocyanate ratio corresponding to an OH:NCO equivalent ratio >1 to 5.

The new process for preparing urethane acrylates of the invention is referred to below as “process of the invention”.

Found not least has been the new use of urethane acrylates of the invention and of urethane acrylates of the invention prepared by the process of the invention as or to prepare materials which are free-radically curable thermally and/or with actinic radiation, this being referred to below as “inventive use”.

Additional subject matter of the invention will become apparent from the description.

### ADVANTAGES OF THE INVENTION

In the light of prior art it was surprising and unforeseeable for the skilled worker that the object on which the present invention was based could be achieved by means of the urethane acrylates of the invention, the process of the invention, and the inventive use.

In particular it was surprising that the urethane acrylates of the invention had a particularly low viscosity. Preferably their viscosity in the DIN 6 flow cup at 23° C. was <500 s, more preferably <450 s, and in particular <400 s.

The urethane acrylates of the invention were preparable easily, economically, and with very good reproducibility from readily available starting products.

The urethane acrylates of the invention were outstandingly suitable as or for preparing new materials free-radically curable thermally and/or with actinic radiation. In that context it was possible to mix them with customary and known additives, especially pigments and flatting agents, without problems and without great expenditure of energy.

The curable materials of the invention were outstandingly suitable for producing new thermoset materials having a very good profile of properties. In particular they were outstandingly suitable as new coating materials, adhesives, sealants, and precursors for moldings and sheets, all free-radically curable thermally and/or with actinic radiation, for producing new thermoset coatings, adhesive layers, seals, moldings, and sheets.

In particular the coating materials of the invention could be applied without problems to a wide variety of substrates by means of rapid methods even without reactive diluent or with only a very small amount of reactive diluents, and without organic solvent or with only a very small amount of organic solvents. In particular it was possible to apply them without problems to coils by the coil-coating method.

The applied coating materials of the invention were free-radically curable thermally and/or with actinic radiation, rapidly and without polymerization-induced contraction, or with only such a low level of polymerization-induced contraction that the desired profile of properties was not affected, or not markedly affected, and gave thermoset coatings of the invention, especially new, glossily clear transparent and flat transparent primer coatings, glossy opaque and flat opaque basecoats, glossily clear transparent and flat transparent topcoats, and glossy opaque and flat opaque topcoats, all having an outstanding profile of properties.

In particular the thermoset coatings of the invention exhibited very good mechanical properties, in particular a high hardness, flexibility, and deformability, strong adhesion to a very wide variety of substrates, especially to coils, and also high chemical resistance and weathering stability. The flat thermoset coatings of the invention exhibited an outstanding flatting effect through to a very attractive silk gloss.

### DETAILED DESCRIPTION OF THE INVENTION

The urethane acrylates of the invention can be activated with actinic radiation. Activation initiates and maintains the free-radical polymerization of the ethylenically unsaturated double bonds present in the urethane acrylates of the invention. Activation can alternatively take place thermally.

Urethane acrylates of the invention contain on average per molecule at least two, in particular at least three, structural units of the general formula I:

R{-X—CH₂—CH(—CH₂—O—(O)—CR¹═CH₂)[—O—C(O)—NH-]}ₙ (I),

In the general formula the index n is a number from 1 to 6 and preferably an integer from 1 to 6. More preferably n is 1, 2 or 3, in particular 1 or 2.

The variables R are each a monovalent to hexavalent, low molecular weight or oligomeric, organic radical.

“Low molecular weight” means that the organic radical R is composed of one structural unit or parent structure. In general the low molecular weight organic radicals R have a molecular weight <1000 daltons.

“Oligomeric” means that the organic radical R is composed of at least 2, in particular at least 3, up to 14, structural units, which may be identical or different from one another. In general the oligomeric radicals R have a number-average molecular weight of 100 to 3000 daltons.

The organic radical R is preferably selected from the group consisting of
substituted and unsubstituted radicals,
radicals free from heteroatoms and containing at least one heteroatom Y,
radicals free from and radicals containing at least one divalent, linking radical R²
radicals consisting of and radicals including at least one radical R³ selected from the group consisting of alkyl, cycloalkyl, and aryl radicals.

The heteroatoms Y are preferably selected from the group consisting of boron, silicon, nitrogen, phosphorus, oxygen, and sulfur. In particular the heteroatoms Y are oxygen atoms.

The divalent, linking radical R² is preferably selected from the group consisting of carboxylic ester, thiocarboxylic ester, carbonate, thiocarbonate, phosphate, thiophosphate, phosphonate, thiophosphonate, phosphite, thiophosphite, sulfonate, amide, amine, thioamide, phosphoramide, thiophosphoramide, phosphonamide, thiophosphonamide, sulfonamide, imide, urethane, hydrazide, urea, thiourea, carbonyl, thiocarbonyl, sulfone, sulfoxide, and siloxane groups.

Examples of suitable substituents are isocyanate-reactive functional groups, preferably selected from the group consisting of hydroxyl groups, thiol groups, and primary and secondary amino groups, halogen atoms, preferably selected from the group consisting of fluorine, chlorine, and bromine, nitrile groups or nitro groups. Hydroxyl groups in particular are used.

The alkyl radicals R³ may be linear or branched. Suitable alkyl radicals R³ derive from alkanes having 2 to 30 carbon atoms in the molecule. Highly suitable alkyl radicals R³ derive from alkanes having 2 to 20 carbon atoms in the molecule, preferably from ethane, n-propane, isopropane, n-butane, isobutane, pentane, isopentane, neopentane, hexane, heptane, octane, isooctane, nonane, decane, undecane, dodecane, tridecane, tetradecane, pentadecane, hexadecane, heptadecane, octadecane, nonadecane, and eicosane, especially ethane, n-propane, n-butane and dodecane.

The cycloalkyl radicals R³ may be monocyclic, bicyclic or polycyclic. The bicyclic and polycyclic cycloalkyl radicals may be linearly annelated, spiroannelated or fused. Suitable monocyclic cycloalkyl radicals R³ derive from monocyclic cycloalkanes having 3 to 10 carbon atoms in the molecule, preferably from cyclopropane, cyclobutane, cyclopentane and cyclohexane, and more preferably from cyclohexane. Suitable bicyclic and polycyclic cycloalkyl radicals derive from bicyclic or polycyclic cycloalkanes having 6 to 20 carbon atoms in the molecule, preferably from cyclohexylcyclohexane, spiro[3.3]heptane, spiro[4.4]nonane, spiro[5.4]decane, spiro[5.5]undecane, hydroindane, decalin, norbornane, bicyclo[2.2.2]octane, and adamantane. In particular the cycloalkyl radicals R³ derive from cyclohexane.

The aryl radicals R³ as well may be monocyclic, bicyclic or polycyclic. The bicyclic and polycyclic aryl radicals R³ may be linearly linked or fused. Suitable monocyclic aryl radicals R³ derive from benzene. Suitable bicyclic and polycyclic aryl radicals derive from bicyclic and polycyclic aromatic compounds having 10 to 30 carbon atoms in the molecule, preferably from biphenyl, terphenyl, naphthalene, phenanthrene or fluorene. In particular the aryl radicals R³ derive from benzene.

Examples of particularly suitable radicals R are n-butyl, lauryl, 1,1-dimethylhept-1-yl, ethane-1,2-diyl, propane-1,3-diyl, butane-1,4-diyl, 2-hydroxypropane-1,3-diyl, radicals of the general formula VII:

—CH₂—CH₂—(—O—CH₂—CH₂—)ₚ— (VII),

in which the index is a number from 1 to 20, or phenyl.

The variable X is an oxygen atom or a —C(O)—O— radical which is linked via the carbon atom to the radical R.

The variable R¹ is a hydrogen atom, a halogen atom, a nitrile group, a substituted or unsubstituted alkyl group having 1 to 6 carbon atoms, a substituted or unsubstituted cycloalkyl group having 3 to 6 carbon atoms, or a substituted or unsubstituted aryl group having 6 to 10 carbon atoms. More preferably the variable R¹ is a hydrogen atom or an unsubstituted alkyl group having 1 to 6 carbon atoms, in particular a hydrogen atom or a methyl group.

The urethane acrylates of the invention preferably further contain in the molecule structural units of the general formula (II):

—X—CH₂—CH(—OH)(—CH₂—O—(O)—CR¹═CH₂) (II),

in which the variables X and R¹ are as defined above. The structural units II are linked to the organic radical R.

The urethane acrylates of the invention are preferably prepared by the process of the invention.

In the process of the invention at least one compound, in particular one compound or two compounds, of the general formula III:

R[—X—CH₂—CH(—OH)(—CH₂—O—(O)—CR¹═CH₂)]ₙ (III),

in which the index n and the variables R and R¹ are as defined above, is or are reacted with at least one, especially one, polyisocyanate having at least 2, preferably 2.5 to 6.5, in particular 2.5 to 5.5 isocyanate groups, in a compound III:polyisocyanate ratio corresponding to an OH:NCO equivalent ratio >1 to 5 and in particular 1.5 to 4.

The compounds of the general formula III are preferably what are called oligomeric glycidyl ester and glycidyl ether acrylates and methacrylates, preferably glycidyl ester acrylates and glycidyl ether acrylates. Particular preference is given to using phenoxyglycidyl ether monoacrylate, lauryl glycidyl ester monoacrylate, and Versatic® acid glycidyl ester monoacrylate, especially Versatic® 10 acid glycidyl ester monoacrylate (neodecanoic acid glycidyl ester monoacrylate), ethylene glycol diglycidyl ether diacrylate, propylene glycol diglycidyl ether diacrylate, butylene glycol diglycidyl ether diacrylate, polyethylene glycol 200 diglycidyl ether diacrylate, polyethylene glycol 600 diglycidyl ether diacrylate, and glycerol diglycidyl ether diacrylate, and also glycerol triglycidyl ether triacrylate.

The compounds of the general formula III are sold for example under the brand name Sartomer® CN131, CN132, CN152 or CN133 (glycerol triglycidyl ether triacrylate) from Sartomer or Atofina, the brand name Doublemer® DM from Double Bond, under the trade names Epoxyester M-600A, 40EM, 70PA, 200PA, 1600PA, and 80MFA from Kyoeisha, under the brand name Laromer® 8765 from BASF Aktiengesellschaft, and under the trade name Monomer ACE (neodecanoic acid glycidyl ester monoacrylate) from Hexion.

Alternatively, as part of the process of the invention, the compounds of the general formula III can be prepared by reacting compounds of the general formula IV:

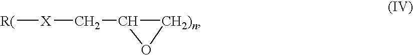

in which the index n and the variables R and X are as defined above, with compounds of the general formula V:

H—O—(O)—CR¹═CH₂ (V),

in which the variable R¹ is as defined above.

In this case it is preferred to set proportions of compound of the general formula IV to compound of the general formula V such as to result in an epoxide group:hydroxyl group equivalent ratio of 0.7:1 to 1.4:1, more preferably 0.8:1 to 1.25:1, and in particular 0.9:1 to 1.1:1.

Examples of particularly suitable compounds of the general formula IV are phenoxyglycidyl ether, lauryl glycidyl ester and Versatic® acid glycidyl ester, especially Versatic® 10 acid glycidyl ester, ethylene glycol diglycidyl ether, propylene glycol diglycidyl ether, butylene glycol diglycidyl ether, polyethylene glycol 200 diglycidyl ether, polyethylene glycol 600 diglycidyl ether, and glycerol diglycidyl ether.

Examples of particularly suitable compounds of the general formula V are acrylic acid and methacrylic acid, especially acrylic acid.

Examples of suitable polyisocyanates are the customary and known polyisocyanates known as paint polyisocyanates, as described for example in detail in the patent applications
DE 199 24 170 A1, column 3 line 61 to column 6 line 68,
DE 103 00 798 A1, page 8, paragraphs [0048] to [0053], page 7 paragraphs [0040] to [0044], and
EP 0 952 170 A1, page 5, Example 1, paragraph [0042].

Accordingly the polyisocyanates, as well as the free isocyanate groups, may also contain blocked isocyanate groups, blocked with the customary and known blocking agents (cf., e.g., also German patent application DE 199 14 896 A1, column 12 line 13 to column 13 line 2) and/or radicals R⁴ containing bonds which can be activated with actinic radiation, especially radicals R⁴ of the general formula VI:

—O—(O)—CR¹═CH₂ (VI),

in which the variable R¹ is as defined above.

The urethane acrylates of the invention can be put to any of a very wide variety of end uses. For example, they can be used as intermediates in organic synthesis. In particular they are used as, or to prepare, innovative materials which are free-radically curable thermally and/or with actinic radiation. The new materials which are free-radically curable thermally and/or with actinic radiation are referred to below for the sake of brevity as “curable materials of the invention”.

It is a particular advantage of the urethane acrylates of the invention and of the curable materials of the invention that they have an advantageously low viscosity even without the addition of organic solvents and/or reactive diluents which can be activated with actinic radiation, and so can be handled and applied without problems.

Accordingly the curable materials of the invention are preferably entirely or substantially free from organic solvents and reactive diluents which can be activated with actinic radiation. “Entirely free” here means that the amount of the reactive diluents and the solvents in the curable materials of the invention in question is so low that it is beneath the detection limits of the customary and known methods of detecting these compounds. “Substantially free” means that the amount of the reactive diluents and the solvents in the curable materials of the invention in question is so low that their performance properties are unaffected by these compounds. This is generally the case for an amount <5%, preferably <3%, and in particular <1% by weight, based in each case on the curable material of the invention.

In the context of the inventive use the curable materials of the invention are employed for producing new thermoset materials.

With particular preference they are used as inventive coating materials, adhesives, sealants, and precursors for sheets and moldings for producing new thermoset coatings, adhesive layers, seals, moldings, and sheets.

With very particular preference they serve as inventive coating materials for producing thermoset coatings of the invention.

In particular the coating materials of the invention are selected from the group consisting of new pigmented and unpigmented, flatted and unflatted primer coating materials and topcoat materials, and also pigmented, flatted and unflatted basecoat materials.

Besides the urethane acrylates of the invention, the coating materials of the invention may further comprise at least one additive in effective amounts. Additives used are preferably those of the kind customary and known within the field of coating materials or paints.

The additives are preferably selected from the group consisting of binders curable physically, thermally, with actinic radiation, and both thermally and with actinic radiation; crosslinking agents; transparent and opaque color pigments, effect pigments, and color and effect pigments; transparent and opaque fillers; nanoparticles; molecularly dispersely soluble dyes; light stabilizers; antioxidants; wetting agents; emulsifiers; slip additives; polymerization inhibitors; thermal crosslinking catalysts; thermolabile free-radical initiators; photoinitiators and photocoinitiators; adhesion promoters; flow control agents; film formation assistants; rheological assistants; flame retardants; corrosion inhibitors; waxes; siccatives, biocides; and flatting agents. These additives are known for example from German patent application DE 199 14 899 A1, page 14 line 36 to page 16 line 63, page 17 line 7 to page 18 line 13, page 18 lines 16 to 21, and page 19 lines 10 to 22 and 30 to 61.

Use is made in particular of photoinitiators and flatting agents. Where the curable materials of the invention are cured using electron beams, there is no need to employ photoinitiators, which is a further particular advantage.

The preparation of the curable materials of the invention has no peculiarities in terms of method but takes place instead preferably by mixing the urethane acrylates of the invention with the above-described additives and homogenizing the resulting mixture using suitable mixing equipment such as stirred tanks, inline dissolvers, rotor/stator dispersers, Ultraturrax devices, microfluidizers, high-pressure homogenizers or nozzle jet dispersers. It is advisable in this context to operate in the absence of actinic radiation.

The coating materials of the invention may serve for producing thermoset coatings of the invention of any of a very wide variety of kinds. By way of example the thermoset coatings of the invention may be new primer coatings, surfacers, antistonechip primer coatings, basecoats, topcoats, and clearcoats.

In particular the thermoset coatings of the invention are selected from the group consisting of glossily clear transparent and flat transparent primer coatings, glossy opaque and flat opaque basecoats, glossily clear transparent and flat transparent topcoats, and glossy opaque and flat opaque topcoats.

Depending on their intended use the curable materials of the invention are applied to temporary or permanent substrates.

For producing sheets and moldings of the invention it is preferred to use customary and known temporary substrates, such as metallic and polymeric belts or hollow bodies made of metal, glass, plastic, wood or ceramic, which are easily removable without damage to the sheets and moldings of the invention.

Where the compositions of the invention are used for producing coatings, adhesive layers, and seals, permanent substrates are employed.

The substrates are preferably
means of land, water, or air transport operated by muscle power, hot air or wind, such as cycles, railroad trolleys, rowboats, sailboats, hot air balloons, gas balloons or sailplanes, and parts thereof,
motorized means of land, water or air transport, such as motorcycles, utility vehicles or motor vehicles, especially automobiles, watergoing or underwater craft or aircraft, and parts thereof,
stationary floating structures, such as buoys or parts of harbor installations,
the interior and exterior of buildings,
doors, windows, and furniture,
PVC floors,
sheets,
paper,
hollow glassware,
small industrial parts, such as nuts, bolts, hubcaps or wheel rims,
containers, such as freight containers or packaging,
coils,
electrical components, such as electronic windings, coils for example,
optical components,
mechanical components, and
white goods, such as household appliances, boilers, and radiators.

The sheets and moldings of the invention may likewise serve as substrates.

In particular the substrates are coils, especially coils made of the customary utility metals, especially bright steel, galvanized, electroplated, and phosphated steel, and aluminum.

In terms of method the application of the curable materials of the invention, particularly of the coating materials of the invention, has no peculiarities but may instead take place by customary and known application methods, such as injecting, spraying, knifecoating, spreading, pouring, dipping, trickling or rolling, for example. Use is made in particular of application methods as are employed in the coil-coating method (in this context cf. Römpp Lexikon Lacke und Druckfarben, Georg Thieme Verlag, Stuttgart, N.Y., 1998, “Coil Coating”, or A. Goldschmidt and H.-J. Streitberger BASF-Handbuch Lackiertechnik, Vincentz Verlag, Hannover, 2002, “4.2.1.2 Brushing, rolling, roller, flood, and pouring methods (paint direct to article)”, pages 521 to 527, and “7.4 Coil Coating”, pages 751 to 756). During application it is advisable to operate in the absence of actinic radiation.

The applied curable materials of the invention, especially the coating materials of the invention, can be cured by free-radical polymerization by means of irradiation with actinic radiation and/or by exposure to thermal energy.

The thermal curing of the applied curable materials of the invention may be accelerated, for example, by exposure to a gaseous, liquid and/or solid, hot medium, such as hot air, heated oil or heated rolls, or to microwave radiation, infrared and/or near (NIR) infrared light. Heating takes place preferably in a forced-air oven or by irradiation using IR and/or NIR lamps.

Curing with actinic radiation may be carried out by means of the customary and known apparatus and methods, as are described, for example, in German patent application DE 198 18 735 A1, column 10 lines 31 to 61, German patent application DE 10202565A1, page 9 paragraph [0092] to page 10 paragraph [0106], German patent application DE 103 16 890 A1, page 17 paragraphs [0128] to [0130], international patent application WO 94/11123, page 2 line 35 to page 3 line 6, page 3 lines 10 to 15, and page 8 lines 1 to 14, or the American U.S. Pat. No. 6,743,466 B2, column 6 line 53 to column 7 line 14.

Curing of the curable materials of the invention can also be carried out in the substantial or complete absence of oxygen.

For the purposes of the present invention oxygen is considered to be substantially absent if its concentration at the surface of the applied curable materials of the invention is <21%, preferably <18%, more preferably <16%, very preferably <14%, with very particular preference <10%, and in particular <6% by volume.

For the purposes of the present invention the oxygen is considered to be completely absent if its concentration at the surface is below the limit of the customary and known detection methods.

The oxygen concentration is preferably ≧0.001%, more preferably ≧0.01%, very preferably ≧0.1%, and in particular ≧0.5% by volume.

The desired oxygen concentrations can be set by means of the measures described in German patent DE 101 30 972 C1, page 6 paragraphs [0047] to [0052] or by the laying-on of sheets.

The resulting thermoset materials of the invention, especially the coatings, adhesive layers, seals, moldings, and sheets of the invention, more especially the coatings of the invention, exhibit numerous particular advantages and so can be employed with an extraordinarily great latitude. For this reason the substrates coated with sheets and/or coatings of the invention, bonded with adhesive layers of the invention, sealed with seals of the invention, packaged with sheets of the invention and/or joined with moldings of the invention likewise exhibit particular advantages, such as a particularly long service life and a high economic value.

In particular the coils of the invention coated with coatings of the invention have a particularly high corrosion resistance. Additionally the adhesion between the coils and the coatings of the invention is outstanding. Because of the outstanding flexibility of the coatings of the invention the coils of the invention can be deformed with no problems. The high hardness and flexibility of the coatings of the invention results in outstanding scratch resistance. On account of the high chemical resistance and weathering stability of the coatings of the invention it is possible to use the parts of the invention produced from the coils of the invention not only in the interior of buildings but also with outstanding effect in the exterior sector. The outstanding flatting effect, which can be increased to the point of a silk gloss, additionally brings with it a particularly appealing esthetic effect.

### EXAMPLES

### Examples 1 to 7

### The Preparation of Urethane Acrylates 1 to 7

Urethane acrylates 1 to 4 of Examples 1 to 4 were prepared using the polyisocyanate Laromer® 9000 from BASF Aktiengesellschaft, which contains 2 acrylate groups and 2 free isocyanate groups.

Urethane acrylates 5 to 7 of Examples 5 to 7 were prepared using the polyisocyanate Desmodur® XP 2410 from Bayer MaterialScience, which is based on hexamethylene diisocyanate.

Urethane acrylates 1 to 7 were prepared in accordance with the following general instructions:

One or two compounds of the general formula III in each case were mixed with one polyisocyanate each in the presence of dibutyltin dilaurate as catalyst (0.02 part by weight in each case per 100 parts by weight of compound III+polyisocyanate). The proportions were adjusted so that the OH:NCO equivalent ratios were >1 (Example 1: 1.86; Example 2: 2.2; Example 3: 4; Example 4: 2; Example 5: 1.5; Example 6: 3.2; Example 7: 2). The reactions were each continued until isocyanate groups were no longer detectable in the reaction mixtures. Table 1 gives an overview of the starting products used, their amounts, and the properties of the resulting urethane acrylates 1 to 7.

TABLE 1
Urethane acrylates 1 to 7: Starting products and properties

| | Starting products: | | Urethane acrylate: | | |
|---|---|---|---|---|---|
| | Compound III | Polyisocyanate | | Mw^{a)} | Viscosity |
| Example | (parts by weight) | (parts by weight) | Functionality | (daltons) | DIN6/23° C. |
| 1 | DM120^{b)} (40) | (20) | 4 | 1.085 | 120 s |
| | CN152^{c)} (40) | | | | |
| 2 | DM120^{b)} (40) | (20) | 4 | 1.083 | 106 s |
| | ACE^{d)} (40) | | | | |
| 3 | ACE^{d)} (80) | (20) | 4 | 1.300 | 95 s |
| 4 | CN152^{c)} (80) | (20) | 4 | 1343 | 115 s |
| 5 | CN152^{c)} (80) | (20) | 4 | 1639 | 380 s |
| 6 | DM120^{b)} (44) | (12.5) | 4 | 984 | 90 s |
| | CN152^{c)} (43.5) | | | | |
| 7 | CN131^{e)} (40) | (20) | >4 | 1341 | 290 s |
| | CN152^{c)} (40) | | | | |

^{a)}mass-average molecular weight (daltons);
^{b)}phenoxyglycidyl ether monoacrylate;
^{c)}lauryl glycidyl ester monoacrylate;
^{d)}neodecanoic acid glycidyl ester monoacrylate;
^{e)}phenoxyglycidyl ether monoacrylate.

Urethane acrylates 1 to 7 had an advantageously low viscosity and could therefore be applied to coils with no problems.

### Examples 8 to 14

### The Production of Clearcoats 1 to 7

Clearcoats 1 to 7 of Examples 8 to 14 were produced using urethane acrylates 1 to 7 of Examples 1 to 7. Urethane acrylates 1 to 7 were knife-coated onto panels of cleaned, galvanized steel and were each exposed to a 65-kilogray dose of electron beams, resulting in clearcoats 1 to 7 having a film thickness each of 18 μm. Measurements were made of their Persoz hardness and their MEK resistance in accordance with the ECCA specification under an applied weight of 1 kg. The results are found in Table 2.

TABLE 2
Persoz hardness and MEK resistance of clearcoats 1 to 7

| | Clearcoat from | Persoz hard- | ECCA MEK resistance |
|---|---|---|---|
| Example | Example | ness (s) | (number of double rubs) |
| 8 | 1 | 88 | 60 |
| 9 | 2 | 63 | 90 |
| 10 | 3 | 91 | 70 |
| 11 | 4 | 77 | 35 |
| 12 | 5 | 153 | 80 |
| 13 | 6 | 110 | >100 |
| 14 | 7 | 180 | 90 |

Clearcoats 1 to 7 exhibit high hardness and a high MEK resistance without the need to add any additives whatsoever. The results of Table 2 also underline the fact that it was readily possible to vary broadly the profile of performance properties of the urethane acrylates. Moreover, clearcoats 1 to 7 were of high gloss, firmly adhering and scratch resistant, and offered a very good protective action with regard to fingerprints.

1. Urethane acrylate composition comprising, on average per molecule, at least two structural units of the general formula I:
R{-X—CH₂—CH(—CH₂—O—(O)—CR¹═CH₂)[—O—C(O)—NH-]}ₙ (I),
wherein
n is a number from 1 to 6;
R is an organic radical selected from the group consisting of monovalent to hexavalent, low molecular mass and oligomeric organic radicals;
X is selected from the group consisting of oxygen atom and —C(O)—O—radical linked by the carbon atom to the radical R; and
R¹ is selected from the group consisting of hydrogen atom, halogen atom, nitrile group, substituted and unsubstituted alkyl group having 1 to 6 carbon atoms, substituted and unsubstituted cycloalkyl group having 3 to 6 carbon atoms, and substituted and unsubstituted aryl group having 6 to 10 carbon atoms.

2. The urethane acrylate composition of claim 1, further comprising at least one structural units of the general formula (II) linked to the organic radical R:
—X—CH₂—CH(—OH)(—CH₂—O—(O)—CR¹═CH₂) (II).

3. The urethane acrylate composition of claim 1, prepared by reacting:
at least one compound of the general formula III:
R[—X—CH₂—CH(—OH)(—CH₂—(O)—CR¹═CH₂)]ₙ (III),
in which the index n and the variables R, X, and R¹ are as defined above; and at least one polyisocyanate having at least two isocyanate groups, in a compound III:polyisocyanate ratio corresponding to an OH:NCO equivalent ratio >1 to 5.

4. The urethane acrylate of claim 3, wherein n is an integer from 1 to 5.

5. (canceled)

6. The urethane acrylate composition of claim 1, wherein the organic radical R is selected from the group consisting of substituted and unsubstituted radicals, radicals free from heteroatoms and radicals having at least one heteroatom Y, radicals free from and radicals having at least one divalent, linking radical R² and radicals consisting of and radicals including at least one radical R³ selected from the group consisting of alkyl, cycloalkyl, and aryl radicals.

7. The urethane acrylates of claim 6, wherein the heteroatom Y is selected from the group consisting of boron, silicon, nitrogen, phosphorus, oxygen, and sulfur.

8. The urethane acrylate composition of claim 6, wherein the divalent linking radical R² is selected from the group consisting of carboxylic ester, thiocarboxylic ester, carbonate, thiocarbonate, phosphate, thiophosphate, phosphonate, thiophosphonate, phosphite, thiophosphite, sulfonate, amide, amine, thioamide, phosphoramide, thiophosphoramide, phosphonamide, thiophosphonamide, sulfonamide, imide, urethane, hydrazide, urea, thiourea, carbonyl, thiocarbonyl, sulfone, sulfoxide, and siloxane groups.

9. The urethane acrylates of claim 1, wherein the variable R¹ is selected from the group consisting of a hydrogen atom and a methyl group.

10. The urethane acrylate composition of claim 3, wherein the at least one compounds of the general formula III is prepared by reacting:
at least one compounds of the general formula IV: and 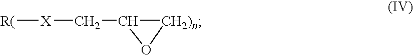
at least one compound of the general formula V:
H—O—(O)—CR¹═CH₂ (V).

11. The urethane acrylate composition of claim 3, wherein the polyisocyanate further comprises radicals selected from the group consisting of blocked isocyanate groups and radicals R⁴ containing bonds which can be activated with actinic radiation.

12. The urethane acrylate composition as claimed in claim 11, wherein radicals R⁴ have the general formula VI:
—O—(O)—CR¹═CH₂ (VI).

13. The urethane acrylate as claimed in of claim 1, which are free-radically polymerizable.

14. The urethane acrylate composition as claimed in claim 11, wherein the actinic radiation is at least one selected from the group consisting of UV radiation and electron beams.

15. A process for preparing urethane acrylates composition as claimed in claim 1, comprising reacting at least one compound of the general formula III:
R[—X—CH₂—CH(—OH)(—CH₂—O—(O)—CR¹═CH₂)]ₙ (III),
with at least one polyisocyanate having at least two isocyanate groups, in a compound III:polyisocyanate ratio corresponding to an OH:NCO equivalent ratio >1 to 5.

16. The process as claimed in claim 15, wherein the OH:NCO equivalent ratio is 1.5 to 4.

17. The process as claimed in claim 15, wherein the at least one compounds of the general formula III are prepared by reacting at least one compounds of the general formula IV: 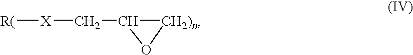
with at least one compounds of the general formula V:
H—O—(O)—CR¹═CH₂ (V).

18. The process of using of the urethane acrylate composition as claimed in claim 1 as a material which is free-radically curable thermally, with actinic radiation or a combination of thermally and with actinic radiation.

19. The process as claimed in claim 18, wherein the material which is free-radically curable thermally, with actinic radiation or a combination of thermally and with actinic radiation is substantially free from organic solvents and reactive diluents which can be activated with actinic radiation.

20. The process as claimed in claim 19, wherein the curable materials is cured under an oxygen-depleted atmosphere.

21. (canceled)

22. (canceled)

23. (canceled)

24. The process as claimed in claim 19, wherein the curable materials serves to produce thermoset materials.

25. The process as claimed in claim 24, wherein the thermoset materials are coatings, adhesive layers, seals, sheets or moldings.

26. The process as claimed in claim 25, wherein the coatings are selected from the group consisting of glossily clear transparent and flat transparent primer coatings, glossy opaque and flat opaque basecoats, glossily clear transparent and flat transparent topcoats, and glossy opaque and flat opaque topcoats.

27. (canceled)
